(19) United States
(12) Patent Application Publication
(10) Pub. No.: US 2024/0277925 A1
(43) Pub. Date: Aug. 22, 2024

(54) HALF-PRESS-FREE DRUG INFUSION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/573,398

(22) PCT Filed: Aug. 5, 2022
(86) PCT No.: PCT/CN2022/110498

§ 371 Date: Dec. 21, 2023

(30) Foreign Application Priority Data
Aug. 6, 2021 (WO) PCT/CN2021/111219
Sep. 10, 2021 (WO) PCT/CN2021/117647
Dec. 9, 2021 (WO) PCT/CN2021/136632

(51) Int. Cl. A61M 5/142

(52) U.S. Cl. CPC A61M 5/14248; A61M 2205/58

(57) ABSTRACT

A half-press-free drug infusion device includes a reservoir configured to accommodate the drug to be infused, provided with a piston and a screw; a driving wheel, connected with screw, driving the screw to push the piston forward by rotation; an infusion needle unit, including an infusion needle and an infusion needle holder. The infusion needle holder is used to keep the infusion needle in place, when leaving the factory, the infusion needle unit is at the intermediate position, the infusion needle is communicated with the reservoir. The half-press-free drug infusion device further includes an adhesive patch, for attaching the infusion device module to the skin surface; and security base, arranged on the bottom of the adhesive patch, provided with an infusion needle accommodating part, which is waterproof and breathable.

### CROSS-REFERENCE TO RELATED APPLICATION

This application claims the priority benefit of PCT application no. PCT/CN2021/117647, filed on Sep. 10, 2021, PCT/CN2021/111219, filed on Aug. 6, 2021, and PCT/CN2021/136632, filed on Dec. 9, 2021. The entirety of the above mentioned patent application is hereby incorporated by reference herein and made a part of this specification.

### TECHNICAL FIELD

The present invention mainly relates to the field of medical instruments, in particular to a half-press-free drug infusion device.

### BACKGROUND

In a healthy person, the pancreas can automatically monitor the amount of glucose in the blood and automatically secrete the required dosage of insulin/glucagon. However, for diabetic patients, the function of their pancreas has been severely compromised, and the pancreas cannot secrete the required dosage of insulin. Therefore, diabetes mellitus is defined as a metabolic disease caused by abnormal pancreatic function, and it is also classified as one of the top three chronic conditions by the WHO. The present medical advancement has not been able to find a cure for diabetes mellitus. Yet, the best the technology could do is control the onset symptoms and complications by stabilizing the blood glucose level for diabetes patients.

A drug infusion device is a medical device that achieves the purpose of disease treatment by continuously infusing drugs into the patient's body. Drug infusion devices are widely used in the treatment of diabetes. Insulin is continuously infused into the patient's skin according to the dosage required by the human body, to simulate the secretory function of the pancreas, thereby maintaining the stability of the patient's blood glucose. The drug fluid is usually stored inside the infusion device. In the existing drug infusion device, the infusion device is usually pasted directly on the patient's body through a medical tape, and the patient operates a remote device for infusion.

When the existing drug infusion device leaves the factory, the infusion needle is in the initial position, and after the user gets the drug infusion device and complete drug filing, the needle holder needed to be half-pressed to the middle position, and then perform the follow-up operation. The operation process is troublesome and lead to bad user experience. Therefore, in the prior art, there is an urgent need for a drug infusion device with good user experience and without half-pressing process.

### BRIEF SUMMARY OF THE INVENTION

The invention discloses a half-press-free drug infusion device, provided with a security base, and an infusion needle accommodating part is set on the security base. The infusion needle accommodating part is waterproof and breathable, so that the infusion needle unit of the drug infusion device can be placed in the intermediate position when it leaves the factory, and the user does not need to half-press the needle hold to the intermediate position, and then to perform follow-up operation, which reduces the user's operation steps, improving the user experience.

The invention discloses a half-press-free drug infusion device that include a reservoir, configured to accommodate the drug to be infused, provided with a piston and a screw; a driving wheel, connected with the screw, driving the screw to push the piston forward by rotation; an infusion needle unit, including an infusion needle and an infusion needle holder, wherein the infusion needle holder is used to keep the infusion needle in place, when leaving the factory, the infusion needle unit is at the intermediate position, the infusion needle communicated with the reservoir; an adhesive patch, for attaching the infusion device module to the skin surface; and security base, arranged on the bottom of the adhesive patch, provided with an infusion needle accommodating part, which is waterproof and breathable.

According to one aspect of the present invention, the security base includes an elastic seal, a waterproof and breathable membrane and a security base body.

According to one aspect of the present invention, the elastic seal and the security base body are integrally formed by injection molding.

According to one aspect of the present invention, the elastic sealing member and the security base body are sealed connected through an interference fit.

According to one aspect of the present invention, the waterproof and breathable membrane is fixed and sealed with the security base body by laser welding or waterproof double-sided tape.

According to one aspect of the present invention, the security base is a hollow elastic sealing member, at least one side of the hollow elastic sealing member is provided with a plurality of dense micro-holes.

According to one aspect of the present invention, the thickness of the security base is gradually decreased from the end close to one end of the adhesive patch to the middle part of the adhesive patch, and forms a plane with the other end of the adhesive patch.

According to one aspect of the present invention, the thickness of the safety base is uniform.

According to one aspect of the present invention, the adhesive patch includes at least one layer of tape and a protective film, the protective film is fixed around the outer contour of at least one layer of tape.

According to one aspect of the present invention, the adhesive patch includes at least two layers of tape.

According to one aspect of the present invention, the adhesive patch further includes at least one layer of protective film.

According to one aspect of the present invention, the Rockwell hardness of the protective film is greater than that of the tape.

According to one aspect of the present invention, the adhesive patch includes a tape and a release paper, the surface of the tape is also provided with dense ventilation holes.

According to one aspect of the present invention, the security base is provided with a third through hole.

According to one aspect of the present invention, the third through hole is tapered.

According to one aspect of the present invention, the infusion device includes an infusion mechanism module and a control mechanism module. The drug reservoir, the drive wheel, and the infusion needle unit are arranged on the infusion mechanism module.

According to one aspect of the present invention, the infusion mechanism module and the control mechanism module are designed separately, and the control mechanism module can be reused.

According to one aspect of the present invention, the infusion mechanism module and the control mechanism module are electrically connected by an electrical contact.

According to one aspect of the present invention, the infusion mechanism module and the control mechanism module are disposed of in one housing, discarded together after a single-use.

Compared with the prior art, the technical solution of the present invention has the following advantages:

In the half-press-free drug infusion device disclosed by the present invention, provided with a security base, and an infusion needle accommodating part is set on the security base. The infusion needle accommodating part is waterproof and breathable, so that the infusion needle unit of the drug infusion device can be placed in the intermediate position when it leaves the factory, and the user does not need to half-press the needle hold to the intermediate position, and then to perform follow-up operation, which reduces the user's operation steps, and as the priming process can be completed during the filling process, avoiding priming waiting, shortening the user's operation period, and significantly improving the user experience.

Furthermore, the infusion needle accommodating part is constituted by an elastic seal, a waterproof and breathable membrane and a security base body, the elastic seal and the security base body are integrally formed by injection molding or sealedly connected through an interference fit, the waterproof and breathable membrane is fixed and sealed with the security base body by laser welding or waterproof double-sided tape, the production process for that is simple, thus reducing the production cost.

Furthermore, the security base is a hollow elastic sealing member, the hollow part is the infusion needle accommodating part, at least one side of the hollow elastic sealing member is provided with a plurality of dense micro-holes, preventing the drug from flowing out, but allow the air to be discharged.

Furthermore, the adhesive patch includes a tape and a release paper, the surface of the tape is also provided with evenly arranged dense ventilation holes, which makes the tape breathable, and can help the water vapor and sweat secreted by the skin of the patient to be discharged, and prevent the water vapor and sweat from soaking the skin and causing the patient's skin to wrinkle.

Furthermore, the adhesive patch includes a tape and a protective film. The protective film can prevent or delay the edge of the tape from curling or warping, thereby preventing the infusion device from being displaced or falling off, which affects the user experience.

Furthermore, the adhesive patch includes at least two layers of tapes, and through the relay action of the tapes, the infusion device is pasted on the surface of the host skin for a long time, thus prolonging the use time of the infusion device.

Further, a tapered hole is provided on the security base, to facilitate guiding an external drug filling device to fill drug.

### BRIEF DESCRIPTION OF THE DRAWINGS

FIG. 1a and FIG. 1b are schematic top views of the drug infusion device according to two embodiments of the present invention.

FIG. 2a and FIG. 2b are schematic views of the control mechanism module according to an embodiment of the present invention.

FIG. 3a is a schematic view of the infusion mechanism module according to an embodiment of the present invention.

FIG. 3b is a side view of the assembly of the control mechanism module and the infusion mechanism module according to an embodiment of the present invention.

FIG. 3c is a schematic top view of the lower case of the infusion mechanism module according to an embodiment of the present invention.

FIG. 3d is a schematic top view of the lower case of the infusion mechanism module according to another embodiment of the present invention.

FIG. 4a and FIG. 4b are schematic views of the internal mechanism module of the infusion mechanism module according to an embodiment of the present invention, respectively.

FIG. 4c is a schematic view of the internal mechanism module of the infusion mechanism module according to another embodiment of the present invention.

FIG. 5 is a schematic view of the elastic conductor according to an embodiment of the present invention.

FIG. 6 is a schematic view of the conductive tower-spring according to an embodiment of the present invention.

FIG. 7 is a partially enlarged view of the part M portion in FIG. 4a according to en embodiment of the present invention.

FIG. 8a and FIG. 8b are schematic views of the driving wheel assembly and the frame before and after assembly according to the embodiment of the present application embodiment, respectively.

FIG. 8c is a schematic view of the shield according to an embodiment of the present invention.

FIG. 9a-FIG. 9d are schematic views of the movable block opened or closed according to an embodiment of the present invention, respectively.

FIG. 10a a schematic view of the adhesive patch according to an embodiment of the present invention.

FIG. 10b a schematic view of each layer of the adhesive patch according to an embodiment of the present invention.

FIG. 10c a schematic view of the cascading sequence of the adhesive patch according to an embodiment of the present invention.

FIG. 10d a schematic view of each layer of the adhesive patch with multiple tapes according to an embodiment of the present invention.

FIG. 10e a schematic view of the cascading sequence of the adhesive patch with multiple tapes according to an embodiment of the present invention.

FIG. 11a is a schematic view of the infusion device according to an embodiment of the present invention.

FIG. 11b is a schematic view of the needle mechanism according to an embodiment of the present invention.

FIG. 11c is a schematic view of the drug storage unit and the cavity according to the embodiment of the present invention.

FIG. 11d is a schematic view of the security base according to an embodiment of the present invention.

FIG. 11e and FIG. 11f are section views of the security base according to other embodiments of the present invention.

### DETAILED DESCRIPTION

As mentioned above, in the prior art, when the drug infusion device leaves the factory, the infusion needle is in the initial position, and after the user gets the drug infusion device and complete drug filing, the needle holder needed to be half-pressed to the middle position, and then perform the follow-up operation. The operation process is troublesome and lead to bad user experience.

In order to solve this problem, the present invention provides a half-press-free drug infusion device, provided with a security base, and an infusion needle accommodating part is set on the security base. The infusion needle accommodating part is waterproof and breathable, so that the infusion needle unit of the drug infusion device can be placed in the intermediate position when it leaves the factory, and the user does not need to half-press the needle hold to the intermediate position, and then to perform follow-up operation, which reduces the user's operation steps, improving the user experience.

Various exemplary embodiments of the present invention will now be described in detail regarding the figures. The relative arrangement of the components and the steps, numerical expressions and numerical values outlined in the embodiments are not construed as limiting the scope of the invention.

In addition, it should be understood that, for ease of description, the dimensions of the various components shown in the figures are not necessarily drawn in the actual scale relationship; for example, the thickness, width, length or distance of certain units may be exaggerated relative to other mechanism modules.

The following description of the exemplary embodiments is merely illustrative and does not limit the invention its application or use. The techniques, methods, and devices are known to those of ordinary skill in the art and may not be discussed in detail. However, such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

FIG. 1a and FIG. 1b are schematic top views of the drug infusion device according to two embodiments of the present invention.

In the embodiment of the present invention, the drug infusion device comprises a control mechanism module 100, an infusion mechanism module 110 and an adhesive patch 120, which will be described separately below. In other embodiments of the present invention, the patch-type drug infusion device may include more parts, which are not specifically limited here.

The patch-type drug infusion device refers to a tubing-free infusion device that is entirely pasted on the user's skin surface by the one piece of adhesive patch 120. And the infusion device is provided with an infusion needle unit 121, integrated on the infusion device, instead of a long tube; therefore, the drug can be directly infused from the reservoir 131 to the subcutaneous tissue through the infusion needle unit 121.

In the embodiment of the present invention, the infusion mechanism module 110 and the control mechanism module 100 are designed separately and connected by a waterproof plug or directly engaged and electrically connected into a whole. Details regarding how the reliability of the electrical connection has been improved when the infusion mechanism module 110 and the control mechanism module 100 are directly engaged and electrically connected into a whole will be described below. The infusion mechanism module 110 can be reused, and the control mechanism module 100 is discarded after a single use, as shown in FIG. 1a. In another embodiment of the present invention, the infusion mechanism module 110 and the control mechanism module 100 are connected by a wire and disposed of inside the same housing 10. Attached to a certain position of the user's skin by the adhesive patch 120, both units will be discarded together after a single use, as shown in FIG. 1b.

The patch-type drug infusion device of the embodiment of the present invention includes a control mechanism module 100, which receives signals or information from a remote device or a body fluid parameter detection device (such as CGM), and controls the infusion device to infuse drug(s) accordingly.

Inside the housing 101 of the control mechanism module 100 are disposed of program modules, circuit board(s) and related electronic units for receiving signals or issuing control instructions, as well as other mechanical units or parts necessary for realizing the infusion function, which is not limited herein. In another embodiment of the present invention, a power supply 133 can also be provided in the control mechanism module. Preferably, in the embodiment of the present invention, the power supply 133 is provided in the infusion mechanism module 110, which will be described below.

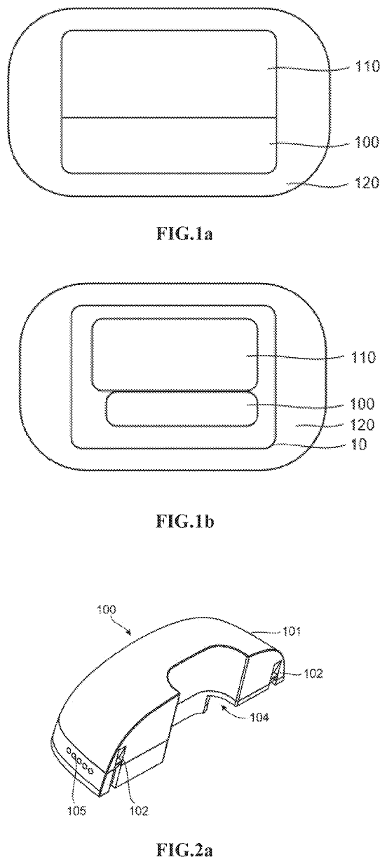

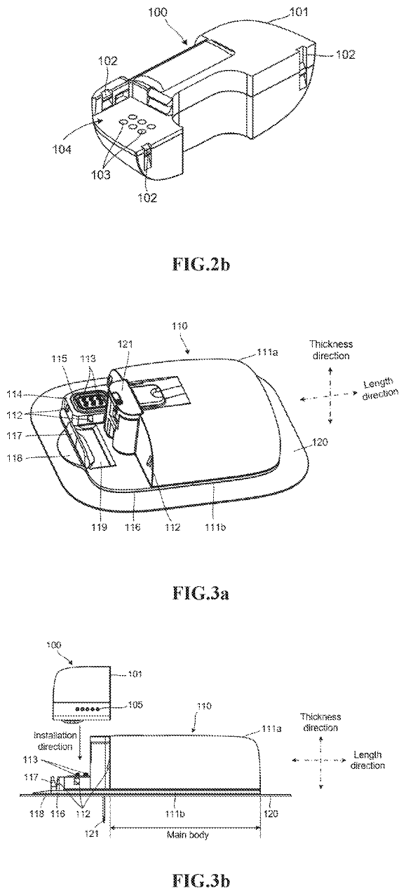

FIG. 2a and FIG. 2b are schematic views of the control mechanism module according to an embodiment of the present invention.

The control mechanism module 100 further includes the first electrical contact 103 exposed on its surface. The first electrical contact 103 is used as a circuit connection terminal to connect the internal circuits provided in the control mechanism module 100 and the infusion mechanism module 110, respectively. The embodiment of the present invention does not specifically limit the position of the first electrical contact 103.

Compared with the plug connector used as a connection terminal in the prior arts, the contact area of the electrical contact is much smaller, which provides more flexibility to the mechanism module design, and can effectively reduce the volume of the control mechanism module. At the same time, these smaller electrical contact can be directly connected to the internal circuit or electrical components. They could also be directly soldered on the circuit board, which helps to optimize the design of the internal circuit and effectively reduce the complexity of the circuit, thereby saving costs and reducing the volume of the infusion device. Furthermore, the electrical contact are exposed on the surface of the control mechanism module 100 to facilitate electrical connection with connection ends on other mechanism modules.

The type of the first electrical contact 103 includes rigid metal pins or elastic conductive members. Preferably, in the embodiment of the present invention, the first electrical contact 103 is a rigid metal pin. One end of the first electrical contact 103 is electrically connected to the connection end provided inside the control mechanism module 100. In contrast, the other end is exposed on the surface of the lower housing 101b. The rest part of the first electrical contact 103 is tightly embedded in the housing 101, thus keeping the internal control mechanism module 100 isolated from the outside.

The elastic conductive member includes conductive spring, conductive silica gel, conductive rubber, or conductive leaf spring. One end of the elastic conductive member is used to electrically connect with the internal connection end in the control mechanism module 100, while the other end is used to connect with other connection ends electrically.

As in an embodiment of the present invention, the first electrical contact 103 is a conductive spring. When the electrical contact are in contact with each other, the elasticity of the conductive spring can enhance the reliability of the electrical connection. Similar to the rigid metal pin, one end of the conductive spring is exposed on the surface of the lower housing 101b. In contrast, the remaining part of the conductive spring is tightly embedded in the housing 101 and electrically connected with internal circuits or electrical components. The connection end disposed inside the control mechanism module 100 can be a conductive lead, a specific part of a circuit, or an electrical element.

It should be noted that the “tightly embedded” in the embodiment of the present invention suggests that there is no gap between the electrical contact and the housing 101, keeping the control mechanism module 100 tightly sealed. The following “tightly embedded” has the same meaning as here.

In another embodiment of the present invention, the first electrical contact 103 is a conductive spring, but it is not tightly embedded in the housing 101. Instead, a sealing element is provided in a groove, both of which are disposed around the area where the first electrical contact 103 is located, thus sealing the electrical contact area and the control mechanism module 100.

In the embodiment of the present invention, the control mechanism module 100 is further provided with the first engaging portions 102, which is used to fasten the second engaging portion 112 disposed on the infusion mechanism module 110 to assemble the control mechanism module 100 infusion mechanism module 110. Details regarding how the mechanism works to enable the electrical connection between the first electrical contact 103 and the second electrical contact 113 will be described below.

The first engaging portion 102 and the second engaging portion 112 include one or more books, blocks, holes, and slots that can be engaged with each other. The positions of the hooks, blocks, holes, and slots can be flexibly adjusted, according to the shape and mechanism module features of the control mechanism module 100 and the infusion mechanism module 110, such as disposed in the interior or on the surface of the corresponding mechanism module, which is not specifically limited herein.

In the embodiment of the present invention, the control mechanism module 100 is further provided with a concave 104 that fits the convex portion 114 disposed at the bottom of the case of the infusion mechanism module 110, which will be described in detail below. Preferably, the first electrical contact 103 is provided in the concave 104, as shown in FIG. 2b.

In the embodiment of the present invention, a buzzer (not shown) is also provided in the control mechanism module 100. When the infusion process starts or ends, the infusion device malfunctions, the drug is exhausted, the control mechanism module 100 issues an error command or receives an error message, etc., the buzzer is used to issue alarm signals, such as sound or vibration, notifying the user to adjust or replace the device in time.

Preferably, in the embodiment of the present invention, the housing 101 of the control mechanism module 100 is provided with a sound-permeable outlet 105 to allow the sound alarm signal from the buzzer to be sent out. In order to achieve a good sealing effect and ensure the normal operation of the buzzer, a waterproof sound-permeable membrane (not shown) is disposed between the sound-permeable outlet 105 and the buzzer. Therefore, the waterproof sound-permeable membrane needs to have a certain porosity to ensure the sound transmission but prevent water molecules penetration.

Compared with the traditional technical solution in which the buzzer is entirely enclosed in the control mechanism module 100, because of the sound-permeable outlet 105, a less loud sound signal emitted from the buzzer would be enough to raise the user's attention, which reduces the energy consumption of the buzzer, thereby optimizing the power consumption configuration of the infusion device and saving production costs.

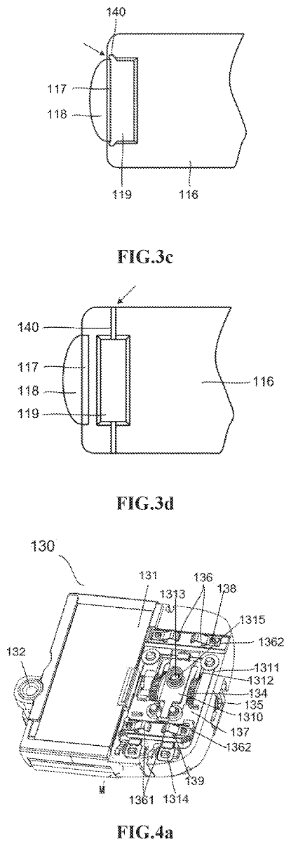

FIG. 3a is a schematic view of the infusion mechanism module 110 according to the embodiment of the present invention. FIG. 3b is a side view of the assembly of the control mechanism module 100 and the infusion mechanism module 110 according to the embodiment of the present invention. FIG. 3c is a schematic top view of the lower case of the infusion mechanism module according to an embodiment of the present invention. FIG. 3d is a schematic top view of the lower case of the infusion mechanism module according to another embodiment of the present invention.

The drug infusion device further includes an infusion mechanism module 110 with a case. A mechanical module, an electric control module, and other auxiliary modules for completing the drug infusion process are provided inside the case, which will be described in detail below. The case of the infusion mechanism module 110 may include multiple parts. As in the embodiment of the present invention, the case of the infusion device includes an upper case 111a and a lower case 111b.

As mentioned above, in the embodiment of the present invention, the infusion mechanism module 110 is provided with the second engaging portions 112, which is used to engaged and fasten the corresponding first engaging portions 102. The positions where the first engaging portions 102 and the second engaging portions 112 are provided correspondingly.

In the embodiment of the present invention, the infusion mechanism module 110 is provided with second electrical contact 113, which are used to press against the corresponding first electrical contact 103 to create an electrical connection between the control mechanism module 100 and the infusion mechanism module 110. The compression between these two corresponding electrical contact disposed on different parts can improve the reliability of the electrical connection. Similar to the first electrical contact 103, one of the second electrical contact 113 also includes a rigid metal pin and an elastic conductive member. Preferably, in the embodiment of the present invention, the second electrical contact 113 is a conductive spring. Similarly, the conductive spring can improve the electrical connection performance. A groove is also arranged around the area where the second electrical contact 113 is disposed, and a sealing member 115 is arranged in the groove. Similarly, the elasticity of the conductive spring can further improve the electrical connection performance.

Preferably, in the embodiment of the present invention, the two ends of the conductive spring have different diameters. And the diameter of the end exposed to the outside of the infusion mechanism module 110 is shorter than that of the end inside the infusion mechanism module 110. In this way, the conductive spring can be held in the case because of the longer diameter; Thus, when the control mechanism module 100 is not installed on the infusion mechanism module 110, the longer diameter of the inner end can prevent the conductive spring from detaching from the infusion mechanism module 110.

The embodiment of the present invention does not limit the position at where second electrical contact 113 is arranged, as long as it can be electrically connected to the corresponding first electrical contact 103. Preferably, in the embodiment of the present invention, the upper case 111a of the infusion mechanism module 110 includes a convex portion 114 where the second electrical contact 113 is disposed, as shown in FIG. 3a. The shape of the convex portion 114 corresponds to that of the concave 104 disposed on the control mechanism module 100, allowing the two portions to tightly fit each other and press the first electrical contact 103 and the corresponding second electrical contact 113 against each other to realize the electrical connection.

In other embodiments of the present invention, the convex portion 114 may be provided on the lower case 111b. When the infusion mechanism module 110 includes an integral case, the convex portion 114 will be a part of the integral case not specifically limited herein. The method of assembling the control mechanism module 100 and the infusion mechanism module 110 to each other includes pressing the control mechanism module 100 on the infusion mechanism module 110 along the thickness direction of the infusion mechanism module 110, thereby engaging the first engaging portion 102 and the second engaging portion 112; or pressing the control mechanism module 100 on the infusion mechanism module 110 along the length direction of the infusion mechanism module 110. Alternatively, the control mechanism module 100 can be pressed along with any angle between the thickness direction and the length direction of the infusion mechanism module 110, making the first engaging portion 102 and the second engaging portion 112 engaged with each other. Preferably, in the implementation of the present invention, the method by which the control mechanism module 100 and the infusion mechanism module 110 are assembled is to press the control mechanism module 100 on the infusion mechanism module 110 along with the thickness direction of the infusion mechanism module 110, making the first engaging portion 102 and the second engaging portion 112 engaged with each other, as shown the installation direction in FIG. 3b.

In the embodiment of the present invention, the lower case 111b of the infusion mechanism module 110 further includes an outward extending portion 116. A block 117 is provided outside the outer extending portion 116, as shown in FIG. 3a. As mentioned above, the control mechanism module 100 is pressed to the engaging position along the thickness direction of the infusion mechanism module 110; thus, block 117 can prevent the control mechanism module 100 from detaching along the length direction of the infusion mechanism module 110, ensuring the normal operation of the infusion device. Obviously, in another embodiment of the present invention, if the control mechanism module 100 is pressed to the engaging position along with other directions, the control mechanism module 100 can also be prevented from detaching from the infusion mechanism module 110 by adjusting the position of the block 117.

It should be noted here that “outer” and “outside” are relative to the main body of the infusion mechanism module 110, where they belong to a concept of the relative position, as shown in FIG. 3a or FIG. 3b. The “outside” below have the same meaning as here.

In the embodiment of the present invention, the outer end of the outer extending portion 116 is also provided with a pressing portion 118 for releasing the blocking effect of block 117. While the user is replacing the infusion mechanism module 110, the control mechanism module 100 can be released from block 117 by pressing the pressing portion 118 with a finger. Then, the user can remove the control mechanism module 100 from the infusion mechanism module 110 with two fingers.

Another embodiment of the present invention can also be provided with an unlocking hole 119 disposed of in the inner side of block 117. While the pressing portion 118 is pressed, a finger can enter the unlocking hole 119, thereby pushing the control mechanism module 100 out to separate the control mechanism module 100 from the infusion mechanism module 110. In the embodiment of the present invention, the unlocking hole 119 is square. The square unlocking hole 119 can facilitate the smooth entry of fingers. In other embodiments of the present invention, the unlocking hole 119 may also have other shapes, which is not specifically limited here.

The lower case 111b of the infusion mechanism module 110 is also provided with one or more crease grooves 140. Two crease grooves 140 are provided on both sides of the unlocking hole 119, as shown in FIG. 3c and FIG. 3d. After the crease groove, 140 is provided, the thickness or width of the lower case 111b at the crease groove 140 (as shown by the arrows in FIG. 3c and FIG. 3d) is reduced. When the user presses the pressing portion 118, the lower case 111b is easily broken at the crease groove 140, and the blocking of the control mechanism module 100 by block 117 is more smoothly released.

Preferably, in the embodiment of the present invention, two crease grooves 140 are provided at the both ends of block 117, respectively, as shown in FIG. 3c. In another embodiment of the present invention, the crease groove 140 is provided on two corresponding lateral sides of the unlocking hole 119, as shown in FIG. 3d.

The drug infusion device further includes an infusion needle unit 121, used for infusing the drug to the subcutaneous tissue.

An adhesive patch 120 is also provided on the bottom of the lower case 111b to attach the infusion device to the user's skin surface.

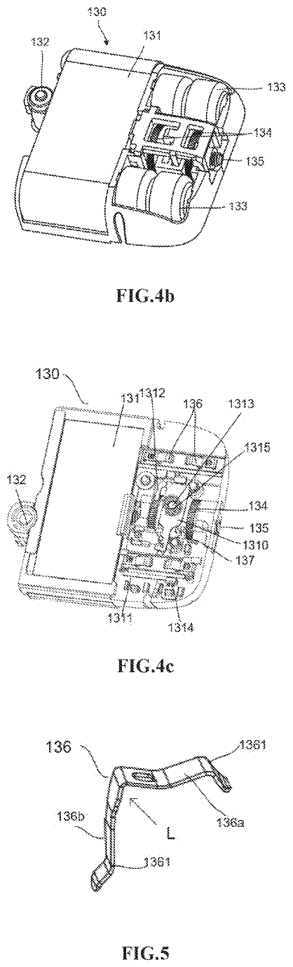

FIG. 4a and FIG. 4b are two schematic views of the internal mechanism module 130 of the infusion mechanism module 110 of the embodiment of the present invention from two perspectives, respectively. FIG. 4c is a schematic view of the internal mechanism module 130 of the infusion mechanism module according to another embodiment of the present invention.

In the embodiment of the present invention, the internal mechanism module 130 includes mechanical units and electronic control units used to realize the infusion function, such as a reservoir 131, a drug outlet 132, a power supply 133, a driving wheel 134, a screw 135, a circuit board (not shown), a driving unit 1310, etc. The movement of the driving unit 1310 drives the driving wheel 134 to rotate, thus making the screw 135 push the piston 312 (as shown in FIG. 14) in the reservoir 131 forward, realizing the drug infusion.

In the embodiment of the present invention, the power supply 133 is a conventional button battery. In other embodiments of the present invention, the power supply 133 may also be other types of batteries, as long as it can meet the requirements for supplying power to the infusion device. Preferably, in the embodiment of this present invention, the type of the power supply 133 is a double-row battery pack; that is, two rows of button batteries are arranged on both sides of the driving wheel 134, respectively, as shown in FIG. 4b. Conventionally, the discharge capacity of button batteries is low. The double-row button battery pack can reduce the discharge level of each battery, thereby extending the service life of the battery. Furthermore, the double-row design of the power supply 133 can make the full use of the internal space and improve the integration within the internal mechanism module in the infusion device.

The infusion mechanism module 110 in the embodiment of the present invention is also provided with a circuit board or multiple three-dimensional circuits coated on the surface of a part of the mechanism module for supplying power to specific structural units. According to the internal arrangement characteristics of the infusion device, the shape and position of the three-dimensional circuit can be flexibly designed, which can make the full use of the internal space of the infusion mechanism module, making the arrangement more compact. The circuit board is a hard/rigid circuit board or a flexible circuit board. Preferably, in the embodiment of the present invention, the circuit board is flexible. The shape of the flexible circuit board is adjustable, allowing it to be flexibly designed according to the internal space of the infusion mechanism module 110. At the same time, multiple connection ends can be provided on the flexible circuit board to be electrically connected to second electrical contact 113, thereby connecting the circuits of the control mechanism module 100 and the infusion mechanism module 110, allowing the infusion device to perform drug infusion function.

An elastic conductor 136 is also provided inside the infusion mechanism module 130. The elastic conductor 136 is electrically connected to the power supply 133, and the specific connection end on the circuit board (or three-dimensional circuit), thereby supplying power to specific structural units.

FIG. 5 is a schematic view of the elastic conductor according to an embodiment of the present invention.

In the embodiment of the present invention, the elastic conductor 136 includes a first elastic conductor 136a and a second elastic conductor 136b, the first elastic conductor 136a is connected with the power supply 133, and the second elastic conductor 136b is connected with the specific connection end on the circuit board (or three-dimensional circuit). At least a protrusion 1361 is provided on the first elastic conductor 136a and the second elastic conductor 136b, which facilitate the point contact connection or the line contact connection between the first elastic conductor 136a and the power supply 133, and also facilitate the point contact connection or the line contact connection between the second elastic conductor 136b and the specific connection end on the circuit board (or three-dimensional circuit), thereby improving the electrical connection reliability between the elastic conductor 136 and the power supply 133, and the specific connection end on the circuit board (or three-dimensional circuit). When the first elastic conductor 136a and the second elastic conductor 136b is flat, during use, it is likely to cause a poor connection between the elastic conductor 136 and the power supply 133 and the specific connection end on the circuit board (or three-dimensional circuit), thereby affecting the use effect. In the embodiment of the present invention, protrusion 1361 may be a linear protrusion formed by bending the first elastic conductor 136a or the second elastic conductor 136b, or it may be several dots or other shapes of protrusions formed by other means on the first elastic conductor 136a or the second elastic conductor 136b. The form and number of the protrusions 1361 on the first elastic conductor 136a or the second elastic conductor 136b can be the same or different. Here, the form, number, and forming method of the protrusions 1361 are not specifically limited, as long as the point contact connection or line contact connection between the elastic conductor 136 and the power supply 133 and the specific connection end on the circuit board (or three-dimensional circuit) can be enhanced, improving the reliability of the electrical connection.

In the embodiment of the present invention, the first elastic conductor 136a and the second elastic conductor 136b are approximately arranged in an “L” shape, the first elastic conductor 136a is approximately parallel to the frame 137, and the second elastic conductor 136b is approximately perpendicular to the frame 137. In other embodiments of the present invention, the first elastic conductor 136a and the second elastic conductor 136b can also be arranged in other shapes, which are not specifically limited here, as long as the power supply 133 and the specific connection end on the circuit board (or three-dimensional circuit) can be electrically connected. An insulating member 1362 is also provided at the junction of the first elastic conductor 136a and the second elastic conductor 136b (as shown at the position L in FIG. 5) to prevent the power unit 1311 from contacting the elastic conductor 136 and causing a short circuit during operation, further stop the infusion mechanism module 130 from working. In the embodiment of the present invention, the insulating member 1362 is formed by printing ink. In other embodiments of the present invention, the insulating member 1362 may also be insulating glue, insulating varnish or insulating material, which is not specifically limited herein.

In the embodiment of the present invention, the elastic conductor 136 may be a sheet metal including the first elastic conductor 136a and the second elastic conductor 136b or may be intergrated by the single first elastic conductor 136a and the single second elastic conductor 136b which are directly electrically connected or indirectly electrically connected with other conductive elements, which is not limited herein. When the elastic conductor 136 is a sheet metal including the first elastic conductor 136a and the second elastic conductor 136b, which not only make the elastic conductor 136 with simple technological process, firm connection between the elastic conductor 136a and the second elastic conductor 136b, but also reduce the volume of the elastic conductor 136, save the material and the raw material and technological cost. When the elastic conductor 136 is intergrated by directly electrically connecting the first elastic conductor 136a and the second elastic conductor 136b or indirectly electrically connected with other conductive elements, the elastic member can be flexibly selected according to the requirements of the specific connecting components to optimize the internal design of the infusion mechanism module.

In the embodiment of the present invention, the frame 137 is further provided with a positioning post 138, the elastic conductor 136 is provided with an opening corresponding to the positioning post 138, and the elastic conductor 136 is sleeved on the positioning post 138 through the opening, so that the elastic conductor 136 is fixed to the frame 137. At the same time, the positioning post 138 is melted by hot melting to further fixed the elastic conductor 136 and to prevent the elastic conductor 136 from shaking due to long-term use or other reasons, which cause the poor electric connection between the elastic conductor 136 and the power supply 133 and the specific connection end on the circuit board (or three-dimensional circuit) and affect the use.

In the embodiment of the present invention, a boss 139 is also provided on frame 137. The boss 139 is located below the first elastic conductor 136a to prevent the protrusion 1361 of the first elastic conductor 136a from being flattened or deformed during long-term use, which results in a poor electrical connection between the first elastic conductor 136a and the specific connection end on the circuit board (or three-dimensional circuit), causing a malfunction. Preferably, the setting position of the boss 139 deviates from the projection position of the protrusion 1361 on the frame 137 and is close to the position post 138. On the one hand, it can prevent the circuit board from being damaged by hard contact or compression between the first elastic conductor 136a and the specific connection end on the circuit board (or three-dimensional circuit). On the other hand, it can ensure the elastic electrical contact between the first elastic conductor 136a and the specific connection end on the circuit board (or three-dimensional circuit).

Similar to the elastic conductive member above mentioned, the type of the elastic conductor 136 includes a conductive spring, a conductive leaf spring, a conductive rubber, a conductive silica gel, etc., which are not specifically limited herein, as long as they can meet the requirements for electrically connecting the power supply 133 to specific connection ends on the circuit board (or three-dimensional circuit). Preferably, in the embodiment of the present invention, the elastic conductor 136 is the conductive leaf spring. Since the infusion mechanism module 110 has a double-row battery pack, the multiple conductive leaf springs are also designed as a double-row pack, as shown in FIG. 4a.

As shown in FIG. 4a, the interior of the infusion mechanism module 110 also includes a drive unit 1310 and a power unit 1311, arranged on the frame 137. Frame 137 is also provided with a rotation shaft 1313, the drive unit 1310 is provided with a through hole, and the drive unit 1310 is sleeved on the rotation shaft 1313 through a through hole for fixing. The elastic conductor 136 can realize a direct electrical connection between the power supply 133 and the specific structural units, which helps to optimize the internal circuit design and reduce the complexity of the internal mechanism module. The driving unit 1310 includes a driving end 1312. One end of the power unit 1311 is connected to the driving unit 1310, and the other end is connected to a specific connection ends on the circuit board or three-dimensional circuit through the conductive platform 1314 so as to further realize the connection with the control module in the control mechanism module 100. The control module in the control mechanism module 100 applies power to the power unit 1311, the drive unit 1310 rotates around the rotation shaft 1313, drives the drive end 1312 forward to push the gear teeth of the drive wheel 134 forward, and causes the infusion device to perform drug delivery. In the embodiment of the present invention, there are two driving ends 1312 and two corresponding power units 1311. In another embodiment of the present invention, there is one driving end 1312 and one power unit 1311, as shown in FIG. 4c.

The infusion mechanism module 110 is also provided with a conductive tower-spring 1315 sleeved on the rotating shaft 1313, with one end abuts the drive unit 1310, and the other end electrically connected to a specific connection end on the circuit board or three-dimensional circuit for fixing the drive unit 1310, at the same time, realize the electrical connection between the driving unit 1310 and specific connection ends on the circuit board or three-dimensional circuit.

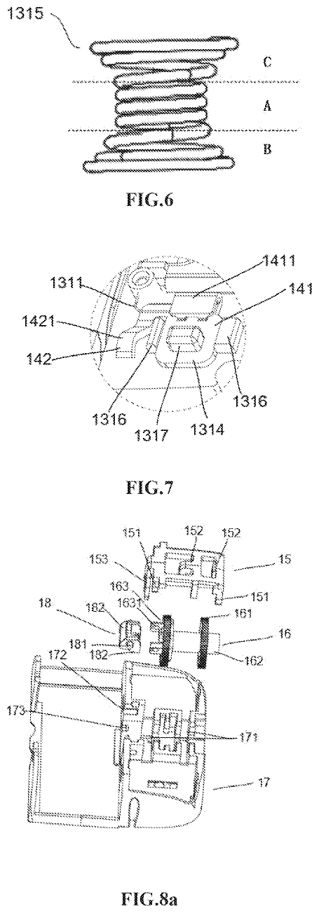

FIG. 6 is a schematic view of the conductive tower-spring according to an embodiment of the present invention.

In the embodiment of the present invention, the conductive tower spring 1315 includes a part A with a small diameter at the central part in the axial direction and a part B and a part C with a large diameter at both ends. The diameter of part A remains the same, and the rotation shaft 1313 is fixed by interference fit, further, fix the drive unit 1310, and at the same time, part B abuts the drive unit 1310 to prevent the drive unit 1310 from shaking due to instability when it is accidentally touched, causing the drive end 1312 to push the drive wheel 134 forward, which affects the accuracy of the infusion. The diameter of the B part and the C part gradually expand towards the two ends in a horn-like shape. When the B part and the C part are respectively electrically connected to the drive unit 1310 and the specific connection ends on the circuit board or three-dimensional circuit, the conductive tower-spring 1315 is compressed; therefore, part B and part C have multiple turns, and at least 2-3 turns of springs are in contact with the driving unit 1310 and the specific connection ends on the circuit board or three-dimensional circuit at the same time, thereby improving the electrical connection reliability of the infusion device. Preferably, in the embodiment of the present invention, parts B and C of the conductive tower-spring 1315 are symmetrical, and the two ends can be assembled at will to avoid assembly misalignment.

FIG. 7 is a partially enlarged view of the part M portion in FIG. 4a according to the embodiment of the present invention.

In the embodiment of the present invention, at least one conductive platform 1314 is provided inside the infusion mechanism module 110. Specifically, when the driving unit 1310 includes two driving arms 1312, there are two corresponding power units 1311 and conductive platforms 1314, as shown in FIG. 4a. When the driving unit 1310 includes one driving arm 1312, there is one corresponding power unit 1311 and one conductive platform 1314, as shown in FIG. 4c. The conductive platform 1314 is electrically connected to the power unit 1311 and the specific terminal on the circuit board or three-dimensional circuit, respectively.

The conductive platform 1314 includes a conductive platform body 141 and a conductive arm 142. The conductive arm 142 is an elastic conductive element. At least one conductive platform protrusion 1421 is provided on the conductive arm 142 to facilitate the connection with specific connection ends on the circuit board (or three-dimensional circuit), improving the electrical connection reliability of the conductive platform 1314 and the specific connection ends on the circuit board (or three-dimensional circuit). When the conductive arm 142 of the conductive platform 1314 is flat, it is likely to cause the poor connection between the conductive platform 1314 and specific connection ends on the circuit board (or three-dimensional circuit) during use, thereby affecting the use effect. In the embodiment of the present invention, conductive platform protrusion 1421 may be a linear protrusion formed by bending conductive arm 142, or it may be several dots or other shapes of protrusions formed by other means on conductive arm 142. Here, the form, number, and forming method of conductive platform protrusion 1421 are not specifically limited, as long as the point contact connection or line contact connection between the conductive platform 1314 and the specific connection ends on the circuit board (or three-dimensional circuit) can be enhanced, improving the reliability of the electrical connection. In the embodiment of the present invention, the conductive platform body 141 further includes an end portion 1411 of the conductive platform body, and the power unit 1311 is an electric drive type linear driver or an electric heating type linear driver, such as a shape memory alloy, which is connected to the conductive platform body 141 by die casting, causing stable connection and highly reliable electrical connection. Specifically, the power unit 1311 is put into the end portion 1411 of the conductive platform body after being folded in integrated and then connected to the end portion 1411 of the conductive platform body 141 by die-casting method further improving the reliability of the electrical connection.

In the embodiment of the present invention, the conductive platform 1314 may be a sheet metal including the conductive platform body 141 and conductive arm 142, or may be formed by the single conductive platform body 141 and the single conductive arm 142 directly electrically connected or indirectly electrically connected with other conductive elements, which is not limited herein. When the conductive platform 1314 is a sheet metal including the conductive platform body 141 and the conductive arm 142, which not only make the conductive platform 1314 with simple technological process, firm connection between the conductive platform body 141 and conductive arm 142, but also reduce the volume of the conductive platform 1314, save the material and the raw material and technological cost. When the conductive platform 1314 is integrated by the single conductive platform body 141 and the single conductive arm 142 directly electrically connected or indirectly connected with other conductive elements, the conductive member can be flexibly selected according to the requirements of the specific connecting components to optimize the internal design of the infusion mechanism module.

Similarly, the conductive platform body 141 and the conductive platform body end portion 1411 can also be sheet metal or integrated by directly or indirectly electrically connecting with other conductive elements, which is not limited herein. The conductive platform 1314 is a sheet metal including the conductive platform body 141, the conductive arm 142 and the conductive platform body end portion 1411, or the two of them can be a sheet metal and then integrated with the third party directly or indirectly through other conductive elements, or all of the three parts are directly integrated or indirectly integrated by electrical connection with other conductive elements, and there is no specific limitation here. The benefits of various forming methods are described above, so it will not be repeated here. Preferably, in the embodiment of the present invention, the conductive platform body 141, the conductive arm 142 and the conductive platform body end portion 1411 are integrally formed.

In the embodiment of the present invention, a plurality of stoppers 1316 are provided on the frame 137 for accommodating and limiting the position of the conductive platform 1314, the frame 137 is further provided with a conductive platform positioning post 1317, the conductive platform 1314 is provided with an opening corresponding to the conductive platform positioning post 1317, and the conductive platform 1314 is sleeved on the conductive platform positioning post 1317 through the opening, so that the conductive platform 1314 is fixed to the frame 137. At the same time, the conductive platform positioning post 1317 is melted by hot melting to further fixed the conductive platform 1314 and to prevent the conductive platform 1314 from shaking due to long-term use or other reasons, which cause the poor electric connection between the conductive platform 1314 and the power supply 133 and the specific connection end on the circuit board (or three-dimensional circuit) and affect the use.

In the embodiment of the present invention, a conductive platform boss (not shown) is also provided on the frame 137, and the conductive platform boss is located below the conductive arm 142 to prevent the conductive platform protrusion 1421 of the conductive arm 142 from being flattened or deformed during long-term use, which results in a poor electrical connection between the conductive arm 142 and the specific connection end on the circuit board (or three-dimensional circuit), causing a malfunction. Preferably, the setting position of the conductive platform boss deviates from the projection position of the conductive platform protrusion 1421 on the frame 137 and it is close to the conductive platform positioning post 1317. On the one hand, it can prevent the circuit board from being damaged by hard contact or compression between the conductive arm 142 and the specific connection end on the circuit board (or three-dimensional circuit). On the other hand, it can ensure the elastic electrical contact between the conductive arm 142 and the specific connection end on the circuit board (or three-dimensional circuit).

Similar to the elastic conductor 136 above mentioned, the type of the conductive platform 1314 includes a conductive spring, a conductive leaf spring, a conductive rubber, a conductive silica gel, etc., which are not specifically limited herein, as long as they can meet the requirements for electrically connecting the power unit 1310 to specific connection ends on the circuit board (or three-dimensional circuit).

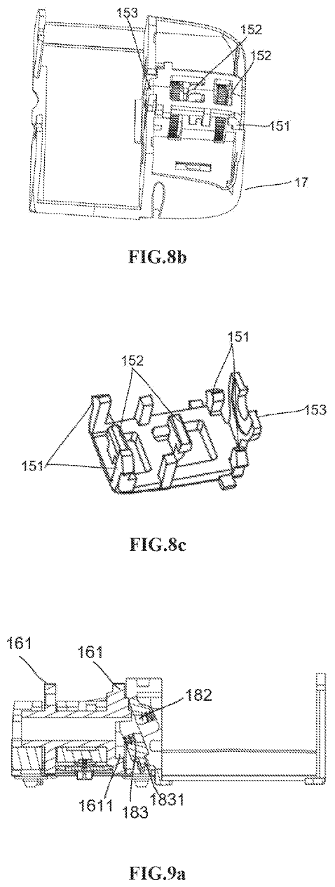

FIG. 8a and FIG. 8b are schematic views of the driving wheel assembly and the frame before and after assembly according to the embodiment of the present application embodiment, respectively. FIG. 8c is a schematic view of the shield according to an embodiment of the present invention.

The driving wheel assembly includes a driving wheel body 16 and a movable block 18. The driving wheel body 16 includes a driving wheel 161, a driving tube 162, which is provided with threads for accommodating the screw 135, and a connecting piece 163 arranged at the end of the driving wheel 161. The connecting piece 163 is provided with a connecting portion 1631, operatively connected with the movable block 18. The movable block 18 is provided with threads to accommodate the screw 135. The movable block 18 is also provided with a movable block connecting rod 181, which is mobily connected with the connecting portion 1631. After the connecting rod 181 is mobily connected with the connecting portion 1631, the movable block 18 can rotate around the connecting rod 181 to open or close the movable block 18. When the movable block 18 is opened, the screw 135 can slide to the driving wheel body 16, and when the movable block 18 is closed, the screw 135 cannot slide to the driving wheel body 16, which will be described in detail below.

The driving wheel assembly further includes a shield 15, and a plurality of shield engaging portions 151 are provided on the shield 15 for engaging with the frame 17. The frame 17 is provided with a frame groove 171 for accommodating the driving wheel body 16 and a plurality of frame engaging portions 172 for engaging with the shield engaging portions 151. After the driving wheel body 16 and the movable block 18 are connected and put into the frame groove 171, the shield 15 is assembled to the frame 17. By the engagement of the frame engaging portion 173 and the shield engaging portion 151, the driving wheel body 16 and the movable block 18 are fixed in the frame 17 to prevent the shaking of the driving wheel assembly and affect the accuracy of drug infusion.

The frame engaging portions 173 and the shield engaging portion 151 include hooks, blocks, holes, and grooves that can be engaged with each other. The position of the engaging portions can be flexibly designed according to the shape and design of the frame 17 and the shield 15, and is not specifically limited here. Preferably, the space formed by the plurality of frame engaging portions 173 and the plurality of shield engaging portions 151 can accommodate the driving wheel body 16 and the movable block 18 to prevent the driving wheel body 16 from shaking.

In the embodiment of the present invention, the shield 15 is also provided with at least one elastic arm 152 for elastically abutting the drive tube 162, which can further fix the drive wheel body 16, improving the stability of the infusion mechanism module 110 and the accuracy of drug infusion. Preferably, two elastic arms 152 are arranged on both sides of one of the driving wheels 161 and abut against the driving tube 162, respectively. As shown in FIG. 8b, the driving wheel body 16 is fixed at multiple points.

The shield 15 is also provided with a baffle 153, and the baffle 153 is provided with a mouth. The main frame 17 is also provided with a card slot 173 on the side close to the reservoir 131 for accommodating the strip (not shown). The strip abuts against the baffle 153 to form a through hole for accommodating the screw 135 and at the same time confine the driving wheel body 16 and the movable block 18 in the frame 17. Preferably, the mouth of the baffle 153 is semi-circular, and the diameter is adapted to the diameter of the screw 135, so as to better accommodate the screw 135 and prevent the screw 135 from shaking greatly under the action of external force.

Preferably, in the embodiment of the present invention, the shield 15 is integrally molded; the technological process is simple, the volume is small, the connection is firm, the material is saved, and the cost is reduced.

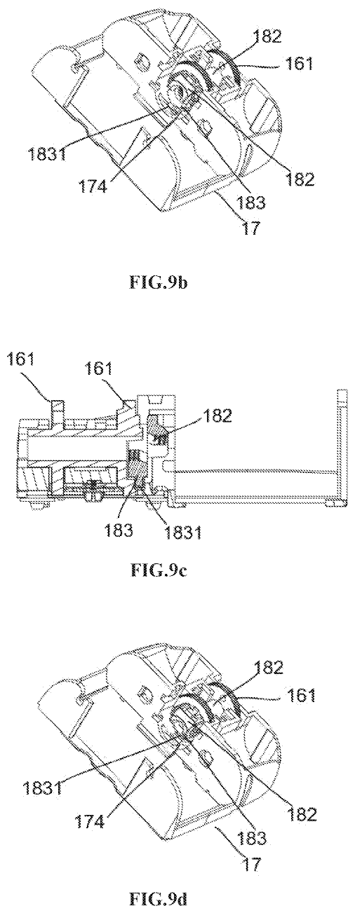

FIG. 9a-FIG. 9d are schematic views of the movable block opened or closed according to an embodiment of the present invention, respectively.

The movable block 18 further includes an upper movable block 182 and a lower movable block 183, and the lower movable block 183 is also provided with a lower movable block end 1831. The movable block 18 opened means that the upper movable block 182 is close to the driving wheel 161, and the lower movable block 183 is far away from the driving wheel 161. The thread in the movable block 18 does not engage with the screw 135. The screw 135 can be smoothly slid in the movable block 18 and the driving tube 162. The movable block 18 closed means that the upper movable block 182 is far away from the driving wheel 161, and the lower movable block 183 is close to the driving wheel 161. At this time, the thread in the movable block 18 is engaged with the screw rod 135, and the screw 135 cannot slide in the movable block 18 and the driving tube 162.

In the embodiment of the present invention, an arc-shaped blocking member 174 is also provided on the frame 17 to limit the position of the lower movable block 183. When the movable block 18 is opened, that is, the upper movable block 182 is close to the driving wheel 161 and the lower movable block 183 is far away from the driving wheel 161, the lower movable block end 1831 of the lower movable block 183 is located outside the arc-shaped blocking member 174, so that the movable block 183 is kept open. At this time, the thread in the movable block 18 does not engage with the screw 135, and the screw 135 can slide smoothly in the movable block 18 and the drive tube 162. When the movable block 18 is opened, the reservoir 131 can be filled with the drug. In the filling process to the reservoir 131, the screw 135 can smoothly move to the end of the driving wheel 161 under the pushing action generated during the filling process, until the filling is completed, no need for the assistance of other parts, which can simplify the complexity of the infusion mechanism module, improve the integration level, save cost, and reduce the volume of the infusion mechanism 110, and at the same time maximizing the actual storage volume of reservoir 131.

When the movable block 18 is closed, that is, the movable block 182 is far away from the driving wheel 161 and the lower movable block 183 is close to the driving wheel 161, the lower movable block end 1831 of the lower movable block 183 is located inside the arc-shaped blocking member 174, and the movable block 18 is kept closed. At this time, the thread in the movable block 18 is engaged with the screw 135, and the screw 135 cannot slide in the movable block 18 and the drive tube 162. When the infusion device 110 performs the drug infusion after the drug is filled, the driving end 1312 of the driving unit 1310 pushes the driving wheel 161 forward, and the movable block 18 rotates with the driving wheel 161. During the rotation, after the lower movable block end 1831 bypasses the arc-shaped blocking member 174, since there is no resistance from the arc-shaped blocking member 174, the lower movable block end 1831 falls into the inside of the arc-shaped blocking member 174. At the same time, since the movable block 18 is kept closed, the screw 135 can only move away from the driving wheel 161 under the pushing action of the rotation of the driving wheel 161 and perform the drug infusion. There is no need to worry about the free movement of the screw 135 due to the engagement failure of the screw 135 with the movable block 18 or the drive tube 162, which will affect the infusion effect or even cause the infusion device to disable.

In the embodiment of the present invention, a notch 1611 is provided on the end face of the driving wheel 16, close to the reservoir 131, the shape is adapted to the lower movable block 183, and is used for accommodating the lower movable block 183. When lower movable block end 1831 bypasses the arc-shaped stopper 174 and falls into the inside of the arc-shaped stopper 174, the lower movable block 183 is accommodated in the notch 1611 of the driving wheel 161, making full use of the space of the driving wheel body, optimizing the internal design of the infusion mechanism module to reduce the volume of the infusion device.

It should be noted here that “inside” and “outside” are relative to the arc-shaped blocking member 174, and belong to the concept of relative position. The positional relationship is as shown in FIG. 9b and FIG. 9d.

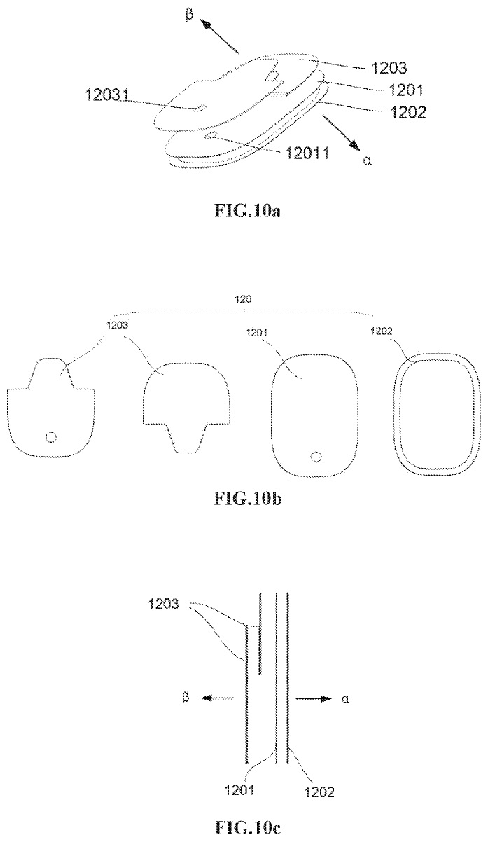

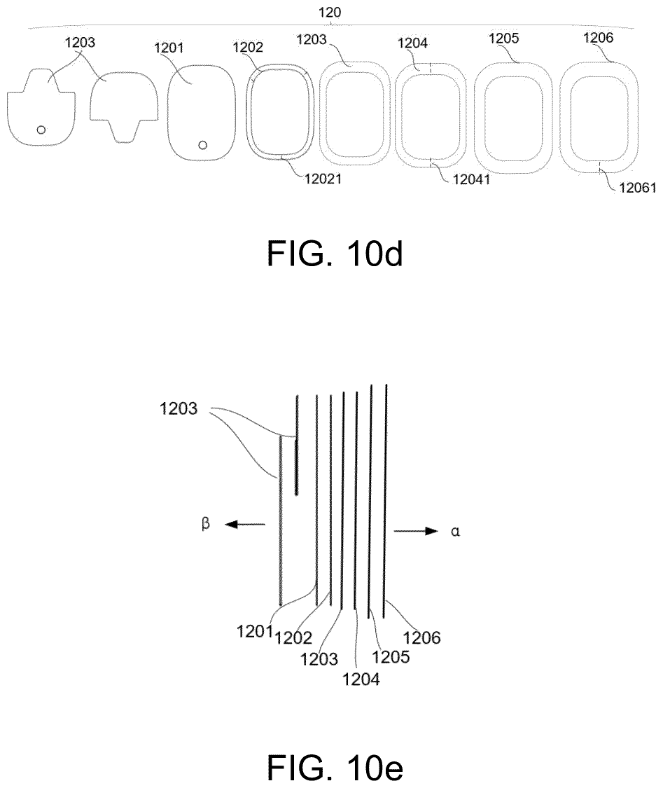

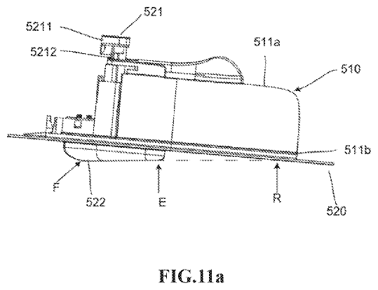

FIG. 10a a schematic view of the adhesive patch according to an embodiment of the present invention. FIG. 10b a schematic view of each layer of the adhesive patch according to an embodiment of the present invention. FIG. 10c is a schematic view of the cascading sequence of the adhesive patch according to an embodiment of the present invention. FIG. 10d a schematic view of each layer of the adhesive patch with multiple tapes according to an embodiment of the present invention. FIG. 10e a schematic view of the cascading sequence of the adhesive patch with multiple tapes according to an embodiment of the present invention.

Adhesive patch 120 comprises a tape 1201, the first side a of the tape 1201 is firmly connected to the infusion mechanism module 110, and the second side B is coated with paste material for pasting the infusion device to the host skin surface. The material of the tape 1201 is one of the following: polyethylene, polypropylene, non-woven or cotton. The tape 1201 is in direct contact with the skin of the host. According to the actual use environment, the above materials can avoid the adverse reactions caused by the tape 1201 contacting the skin for a long time. In order to adapt to the host skin movement, such as tortuous, stretching, etc., the thickness of the tape 1201 is extremely thin, for example, about 1 μm˜500 um. Thin tape can cause other problems, such as static electricity from the host skin, or intense skin movement can cause the edges of the tape to warp. Once the edge of the tape warps, the area of the warped edge of the tape 1201 will gradually increase with the increase of the use time, resulting in the decrease of the adhesion force between the tape 1201 and the skin, which may lead to the displacement or shedding of the infusion device, affecting the user experience.

A protective film 1202 is added on the outer edge of the first a face of the tape 1201, and the Rockwell hardness of the protective film 1202 is greater than that of the tape 1201. Preferably, in the embodiment of the invention, the Rockwell hardness of the protective film 1202 is 80 HRM˜100 HRM.

Preferably, in the embodiment of the invention, the protective film 1202 is one of polycarbonate, polyamide, polyformaldehyde, polyphenyl ether, polyester, polyphenylene sulfide and polyaryl ester.

In a more preferred embodiment of the invention, the protective film 1202 is polyethylene terephthalate (PET), and its Rockwell hardness is 90 HRM˜95 HRM.

The outer contour of the protective film 1202 is compatible with the outer contour of the tape 1201. Here, the outer contour size, bending radius, shape and other parameters of the protective film 1202 are consistent with the outer contour parameters of the tape 1201, so that every part of the outer contour of the tape 1201 can be fitted with the protective film 1202.

In the preferred embodiment of the invention, the thickness of the protective film is 10 um˜500 um.

In the preferred embodiment of the invention, the protective film 1202 is annular; the hollow and annular protective film 1202 can be fitted to the first side a of the tape 1201 comprehensively, and no interference with the infusion device. Secondly, the inner contour of the annular protective film is consistent with the outer contour, which is more beautiful and enhances the user experience.

In the embodiment of the invention, the tape 1201 is provided with a first through hole 12011, and the position of the first through hole 12011 corresponds to the infusion needle 121 of the infusion device, which is used for the infusion needle 121 to pierce the skin of the host.

The second side B of the tape 1201 is also provided with at least one layer of release paper 1203, as the release paper 1203 can prevent the adhesive material of the second side B of the tape 1201 from adhering and can contaminating.

In the preferred embodiment of the invention, the release paper 1203 is a single silicon release paper, and its peeling force is 30 g˜50 g.

In the embodiment of the invention, the release paper 1203 is provided with a second through hole 12031, and the position of the second through hole 12031 corresponds to the first through hole 12011, so that the infusion needle 121 of the infusion device can pass through the first through hole 12011 and the second through hole 12031 to pierce the host skin.

For the convenience of users to peel off release paper 1203 and tape 1201, and save space, the release paper 1203 is preferred to be two layers, and the peel openings are relative to each other, and the peel openings of one layer are bent outwardly and covered by the peel openings of the other layer. Combined with the above, adhesive patch 120 from side a to B is protective film 1202, tape 1201 and release paper 1203 in sequence.

In another embodiment of the present invention, the adhesive patch includes at least two layers of tape. Except that the first layer of tape is fixedly connected to the lower case of the infusion device, the other layers of tape are laminated and sleeved on the infusion device, through the relay action of the tapes, the infusion device can be stick to the surface of the user's skin for a long time, prolonging the use time of the infusion device. The outer contour of the outer tape (away from the skin) completely covers the outer contour of the inner tape (closer to the skin). It should be noted that the complete coverage includes the outer contour of the outer tape and the inner tape are complete overlapped, that is, the areas of the inner and outer tapes are equal, and it also includes the outer contour of the outer tape extends outward along the outer contour of the inner tape, that is, the area of the outer tape is larger than the area of the inner tape.

It should be further explained that, except for the first layer of tape, the remaining layers are all overlaid on the infusion device. Therefore, in the embodiments of the present invention, unless otherwise specified, the “Cover” or “Area” mentioned in the specification is refers to the relationship or area of the inner and outer tapes protruding from the infusion device.

When the outer tape completely covers the adjacent inner tape, after tearing off the outer tape, the exposed inner tape is in a brand new state, free of dirt, improving user experience. Preferably, the area of the outer tape is greater than the area of the adjacent inner tape. Because the outer tape is curled, warped or contaminated first, after tearing off the outer tape, it can be ensured that the exposed inner tape is in a brand new state, further improving the user experience.

In another embodiment of the present invention, the outer tape at least partially covers the inner tape, and the at least partial cover mentioned here includes the outer contour of the inner tape completely protruding from the outer contour of the outer tape. At this time, although the outer contour of the inner tape will be curled or warped to a certain extent first, when the position of the curled or warped edge extends to the position where the outer tape overlaps, the inner tape will stop or suspend warping or curling due to the protective of the outer tape, so as to extend the use time of the infusion device.

At least partially covers also includes that the inner and outer tapes have a part of the outer contour protruding from each other. As mentioned above, when the part of the outer contour of the outer tape protruding from the outer contour of the inner tape curled or warped first, after tearing the outer tape, the part of the inner tape corresponding to the outer layer is in a new state; and when the part of the outer contour of the inner tape protrudes from the outer contour of the outer tape curled or warped first and extend to the position where the outer tape overlaps, the inner tape will stop or suspend warping or curling due to the protective of the outer tape. In both cases, the use time of the infusion device can be extended to a certain extent.

The adhesive patch also includes a layer of protective film, which is set on the outermost layer of tape. The outer contour is compatible with that of the outermost layer of tape to prevent curling or warping of the outermost layer of tape and further extend the use time of the infusion device.

In another embodiment of the present invention, the adhesive patch 120 includes a plurality of tapes and a plurality of protective films, and every two tapes are separated by a protective film to prevent adhesion between adjacent tapes. At the same time, it is convenient to separate the tapes. The tape of the same layer is compatible with the outer contour of the protective film, as shown in FIG. 10d and FIG. 10e, the third tape 1205 and the third protective film 1206 have the same outer contours, and the areas are equal; the second tape 1203 and the second protective film 1204 have the same outer contours, and the areas are equal. The “compatible with” mentioned here means that the outer contour size, bending radius, shape and other parameters of the protective film are consistent with the outer contour parameters of the tape, so that every part of the outer contour of the tape can be attached to the protective film.

In the process of use, when the outermost protective film, that is, the third protective film 1206, loses its protective effect due to falling off and other reasons, tear off the third protective film 1206; continue to use for a period of time, when the outermost tape is curled or warped, that is, when the third tape 1205 is curled or wrapped, tear off the second protective film 1204 and the third tape 1205 together, and the second tape 1203 is exposed. Continue to use for a period of time, when the second tape 1203 is curled or warped, tear off the first protective film 1202 and the second tape 1203 together, the first tape 1201 is exposed, extending the time that the infusion device is stabilized sticked on the surface of the user's skin and improving the user experience. Through the relay of multiple tapes and the protection of the protective films, the infusion device is stuck on the surface of the user's skin for a long time, prolonging the use time of the infusion device.

It should be noted that in the embodiment of the present invention, the multiple tapes and the multiple protective films are not limited to three layers, they may be two layers, or more layers, and they can be choosed according to the requirements of the infusion device need to past on the surface of the user. The outermost layer also can not be provided with a protective film, and the use time of the infusion device can also be prolonged through the layer-by-layer relay of the tapes. Preferably, the outermost layer is provided with a protective film, which can prevent curling or warping of the outermost tape, and further prolong the use time of the infusion device.

It should also be noted that the position and size relationship of the adjacent inner and outer tapes and protective films are consistent with the aforementioned multilayer tapes without protective film, that is, the outer contour of the outer tape and the protective film can completely cover the inner tape or can at least partially cover the outer contour of the inner tape and the protective film. The corresponding beneficial effects are also as described above. At the same time, since there is a protective film between every two tapes, when the areas of the inner and outer tapes and the protective film are not equal, when the outer tape is torn off through the inner protective film, the difference in area between the inner and outer protective film and the tape can be used to easily tear off the inner protective film and outer tape without affecting the inner tape.

In the embodiment of the present invention, the protective film is also provided with at least one tearing portion, as shown by 12021, 12041 and 12061 in FIG. 10d, such as notches, perforated tear lines, or break seams, etc., to facilitate separating the protective film from the inner tape and tear off the outer tape at the same time. Preferably, 2-3 tearing portion s are provided on the inner protective film to facilitate tearing off the protective film and the adjacent outer tape from different positions, so as not to affect the inner tape. There is only one tearing portion on the outermost protective film, which not only facilitates the tearing of the outermost layer of adhesive tape, but also prevents the outermost tape from curling or warping to the maximum extent and prolongs the use time of the infusion device. It should be noted that the specific form of the tearing portion is not limited in the embodiment of the present invention, as long as the outer tape can be easily torn off.

In another embodiment of the present invention, no tearing part is provided on the outermost protective film to prevent users from habitually tearing off the outermost protective film, or opening the outermost protective film or falling off from the outermost tape due to accidental touch, which will lose its protective effect on the outermost tape.

In the embodiment of the present invention, except that the first tape is pasted on the lower case of the infusion device, and the area is adapted to the area of the lower case of the infusion device, the adaptation mentioned here refers to the first tape can cover the area of the lower case, and at the same time extend from the outer contour to a certain distance, so that the infusion device can better stick to the skin. Other tapes and protective films are annular, laminated and sleeved on the infusion device, and do not interfere with the infusion device. In addition, the inner and outer contour of the annular tapes and protective films are consistent with each other, which is more beautiful and can enhance the user experience.

In the embodiment of the present invention, the materials of each tape and protective film are as described above, and will not be repeated here. The materials of each tape and protective film can be the same or different, and it can be selected according to actual needs.

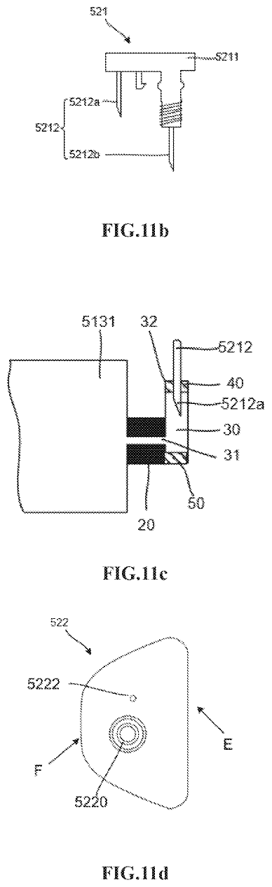

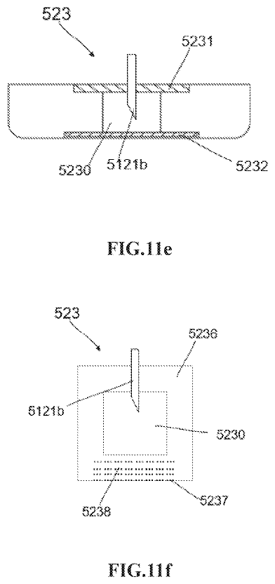

FIG. 11a is a schematic view of the infusion device according to an embodiment of the present invention. FIG. 11b is a schematic view of the needle mechanism according to an embodiment of the present invention. FIG. 11c is a schematic view of the drug storage unit and the cavity according to the embodiment of the present invention. FIG. 11d is a schematic view of the security base according to an embodiment of the present invention. FIG. 11e and FIG. 11f are section views of the security base according to other embodiments of the present invention.

As shown FIG. 11a, the infusion device of the embodiment of the present invention includes control mechanism module (not shown), infusion mechanism module 510, adhesive patch 520 and security base 522, the infusion mechanism module includes the above mentioned mechanical unit, control unit, such as, reservoir, driving wheel etc., and further includes the infusion needle unit 521, which is provided on the upper case 511a, and the adhesive patch 522 is provided on the bottom of the lower case 511b, the security base is provided on the bottom of the adhesive patch 520. The infusion needle includes an infusion needle holder 5211 and an infusion needle 5212, the infusion needle base 5211 is used to fixedly place the infusion needle 5212, making the drug flow infused under the skin of the patient.

As shown in FIG. 11b, the infusion needle 5212 is fixedly placed on the needle holder 5211. The infusion needle 5212 is a U-shaped steel needle. The infusion needle 5212 includes a front end 5212a and a rear end 5212b, both of which extend out of the needle holder 5211. The front end 5212a is used to communicate with the opening 20 of the reservoir 5131, while the rear end 5212b is used to inserted under the skin of the patient.

As shown in FIG. 11c, the upper case 511a is further provided with a cavity 30 including a first outlet 31 and a second outlet 32. The first outlet 31 is in sealed communication with the opening 20. Here, the sealed communication means that the cavity 30 and the reservoir 5131 are in communication with each other through the opening 20 and the first outlet 31 without drug leaking. The second outlet 32 is sealed by an elastic seal 40. When the front end 5212a pierces the elastic seal 40, the infusion needle 5212, the cavity 30, the opening 20 and the reservoir 5131 are in communication. Therefore, the drug can enter the infusion needle 5212 from the reservoir 5131 to the rear end 5212b or be infused under the skin of the patient.

Please continue to refer to FIG. 11a, FIG. 11b and FIG. 11c, in the embodiment of the present invention, the needle holder 5211 has three positions: an initial position, an intermediate position, and a working position. Among them, the intermediate position is set between the initial position and the working position. Since the infusion needle 5212 is fixed on the needle holder 5211, in the embodiment of the present invention, the needle unit also has the above three positions.

Preferably, in the embodiment of the present invention, when the needle holder 5211 is at initial position, the front end 5212a does not pierce the elastic seal 40, so it is not communicated with the second outlet 32. When the needle holder 5211 reaches the intermediate position or the working position, the front end 5212a pierces the elastic seal 40 communicating with the second outlet 32. At this time, the drug, along the cavity 30 and the infusion needle 5212, flows from the reservoir 5131 to the rear end 5212b.

At the beginning, the needle holder 5211 is at the initial position with the infusion needle 5212 non-communication with the reservoir 5131. Therefore, the cavity of the infusion needle 5212 is full filled with air. When leaving the factory, the non-communication between the infusion needle 5212 and the reservoir 5131 can improve the flexibility of the setting position of the needle unit, which optimizes the architecture design of the infusion device.

After drug filling, press the needle holder 5211 to the intermediate position, that is, the needle holder 5211 is half pressed, at that time, the front end 5212a pierces the elastic seal 40 in order to make the infusion needle 5212 communicate with the reservoir 5131 to balance the internal and external pressures. At this time, when the infusion unit is activated, the drug can flow, along the infusion needle 5212, to the rear end 5212b, to prime the fluid path and exhaust the air in the cavity of the infusion needle 5212. When the needle holder 5211 reaches the middle position, the rear end 5212b will protrude from the adhesive patch 520. When priming the fluid patch, in order to completely expel the air in the infusion needle 5212, a small amount of drug from will be flowed out from the needle rear end 5212b. As shown in FIG. 16d, an infusion needle accommodating part 5220 is provided on the security base 522, the position is corresponded to the rear end 5212b, for accommodating the rear end 5212b protruding from the adhesive patch 520, preventing the rear end 5212b from stabing the user, and at the same time, accommodating the drug flowed out from the rear end 5212b when priming, preventing the drug being contaminated due to the drug flowed out and exposed in the air, before the back end 5212b pierced under the skin. If contaminated drugs are infused into the body, it may cause safety risks.

As mentioned above, the adhesive patch includes a protective film, tape and release paper. The tape and the release paper are respectively provided with a first through hole and a second through hole for the infusion needle to pass through. In the embodiment of the present invention, the diameter of the first through hole and the second through hole is smaller than the diameter of top opening of the infusion needle accommodating part 5220, preventing the drug contained in the infusion needle accommodating part 5220 from overflowing from the opening of the infusion needle accommodating part 5220 and infiltrating into the tape, due to shaking or other reasons, which will cause the poor adhesiveness of the tape, and the infusion device will be easily detached from the skin, affecting the user experience. The surface of the tape is also provided with evenly arranged dense ventilation holes, which makes the tape breathable, and can help the water vapor and sweat secreted by the skin of the patient to be discharged, and prevent the water vapor and sweat from soaking the skin and causing the patient's skin to wrinkle.

When the priming is over, the release paper of the adhesive patch 520 is torn off, the security base 522 is removed at the same time, the infusion device is pasted on the user's skin, and then the needle holder 5211 reaches the working position, the rear end 5212b pierces the skin to infusion the drug into the body.

The security base 522 is also provided with a third through hole 5222, which is used to fill the reservoir 5131 with drug, and the position is corresponded to the drug inlet 50, as shown in FIG. 16c. Preferably, the third through hole 5222 is tapered to facilitate guiding an external drug filling device, such as a syringe, to be inserted vertically into the reservoir 5131. The part with smallest diameter of the syringe is compatible (except the needle of the syringe) to the bottom opening (the opening with bigger diameter) of the tapered third through hole, and is used to limit the syringe and prevent the syringe from being inserted too deep or too shallow, which affects the effect of drug infusion. Correspondingly, the tape and release paper of the adhesive patch 520 are provided with a corresponding fourth through hole (not shown), so that the needle of the syringe could pass through the third through hole 5222 and the fourth through hole to fill drug.

As shown in FIG. 11a, the thickness of the security base 522 is gradually decreasing from the end close to one end of adhesive patch 520 to the middle part of adhesive patch 520, that is, gradually decreasing from the F end to the E end, and forms a plane with the other end, R end, of the adhesive patch 520, so that the infusion device can be stably placed on the desktop or other places during priming. Compared with the security base with an area similar to that of the lower case 511b, the trapezoidal security base 522 with a variable thickness can reduce the weight and volume of the entire infusion device, which is conducive to the miniaturization of the infusion device; Compared with the security base with uniform thickness with an area similar to that of the rear end 5212b. Since the two ends of the infusion device have the same height difference relative to the security base, it cannot be placed on the table stably, and it may be shaked by accidentally touched, which causes the drug in the reservoir 5131 or the infusion needle accommodating portion 5220 to shake and affects the use effect. The security base 522 with a variable thickness can form a plane with the lower case 511b of the infusion device. So it can be stably placed on the desktop.

Continuing to refer to FIG. 11d, the security base 522 is trapezoidal, that is, the bonding surface with the adhesive patch 520 is trapezoidal, the short side of the trapezoid is the thicker end F, and the long side of the trapezoid is the thinner end E. The F end corresponds to the unlocking hole of the infusion device (as shown in FIG. 3a 119), the length of the F end is less than the length of the unlocking hole, and the length of the E end is smaller than the width of the infusion device. Preferably, the security base 522 is in the shape of an equilateral trapezoid, which makes the infusion device as small as possible while making the infusion device more stable on the desktop during priming.

In the preceding embodiment, when the drug infusion device leaves the factory, the infusion needle is in the initial position. After drug filing, the needle holder needs to be half-pressed to the intermediate position, and then perform the follow-up operation, leading to bad user experience. If the drug infusion device is half-pressed to the intermediate position when leaving the factory, at that time, the front end 5212a pierces the elastic seal 40 making the infusion needle 5212 communicated with the reservoir 5131. When the user starts to fill the drug, due to the resistance of the piston in the reservoir, the drug enter the infusion needle 5212, reach the rear end 5212b of the infusion needle along the infusion needle 5212, and then reach the infusion needle accommodating part 5220. After filling the infusion needle accommodating portion 5220, the drug will be filled into the reservoir. However, as the infusion needle accommodating part 5220 isn't waterproof and breathable, it can't allow the passage of air while prevent the passage of fluid, the air cannot be exhausted during the drug filling process. Since the security base 522 is disposed under the adhesive patch 520, the sealing between the infusion needle accommodating part 5220 and the lower casing 511b is considered poor. Therefore, during the process the drug filled into the infusion needle accommodating part 5220 first and further filled into the reservoir, part of the drug will overflow from the infusion needle accommodating part 5220. the drug is wasted on one hand, and on the other hand, the overflowed drug infiltrate the adhesive patch 520, affecting the use effect of the adhesive patch 520, thereby affecting the service life of the drug infusion device.

Therefore, in another embodiment of the present invention, it discloses a drug infusion device that can hold the infusion needle in the intermediate position when it leaves the factory, as shown in FIG. 11e. The security base 523 includes an elastic seal 5231, a waterproof and breathable membrane 5232 and a security base body 5234, surrounded to constitute the infusion needle accommodating part 5230. When the infusion needle is in the intermediate position, the rear end 5121b of the infusion needle pierces the elastic seal 5231, and the elastic seal 5231 wraps the rear end 5121b of the infusion needle, which can prevent the drug from overflowing from the infusion needle accommodating part 5230, thereby avoiding drug waste or affecting the adhesive patch. In addition, the air in the fluid path can also be exhausted from the waterproof and breathable membrane 5232 to avoid potential safety hazards.

In the embodiment of the present invention, since the infusion needle unit is in the intermediate position when it leaves the factory, the infusion needle 5212 is in communication with the reservoir 5131. When the user starts to fill the drug, the resistance of the piston in the reservoir causes the drug to enter the infusion needle 5212 first, along the infusion needle 5212 to the rear end 5212b of the infusion needle, and then to the infusion needle accommodating part 5230, after the infusion needle accommodating part 5230 is filled, the drug is filled into the reservoir. Due to the sealing effect of the elastic seal 5231, the waterproof and breathable membrane 5232 and the security base body 5234, the pressure in the infusion needle accommodating part 5230 becomes larger than the pressure in the reservoir. The drug will not overflow from the infusion needle accommodating part 5230, and the air in the fluid path can also be exhausted from the waterproof and breathable membrane 5232, that is, the priming process can be completed during the user's filling process, shortening the user's waiting time, further improving user experience.

In the embodiment of the present invention, the elastic seal 5231 is made of silicone, rubber, resin, etc. The security base body 5234 is PP, PC, or PE, etc. The elastic seal 5231 and the security base body 5234 are integrally formed by injection molding. Waterproof and breathable membrane 5232 is fixed and sealed with the security base body 5234 by laser welding or waterproof double-sided tape. In another embodiment of the present invention, the elastic sealing member 5231 and the security base body 5234 are connected through an interference fit.

In yet another embodiment of the present invention, the security base 523 is a hollow elastic sealing member. As shown in FIG. 11f, the elastic sealing member is a cube, a rectangular solid, a cylinder, or other structures, including a special-shaped body structure. One side 5236 of the security base 523 is used to wrap the rear end 5121b of the infusion needle, and at least one other side 5237 of the security base 523 is provided with a plurality of dense micro-holes 5238. The diameter of the micro-holes 5238 is much smaller than that of a general water droplet, which can prevent the drug from flowing out but can allow the air to be discharged. The hollow part is the infusion needle accommodating part 5230 for accommodating the infusion needle and the drug.

In the embodiment of the present invention, the thickness of the security base 523 can be gradually changed like the security base 522, and a base with equal thickness, or with arbitrarily changed thickness. Since the user can complete the priming process during the drug filling process, the infusion device does not need to be stably placed on the table or other places for priming. Due to the sealing effect of the infusion needle accommodating part 5230, the infusion needle is in the intermediate position, that is, before the security base 523 is removed, the needle accommodating portion 5230 is always filled with drug, and there is no fear of the drug shaking or the drug overflowed due to shaking.

As a summary, the present invention discloses a half-press-free drug infusion device, provided with a security base, and an infusion needle accommodating part is set on the security base. The infusion needle accommodating part is waterproof and breathable so that the infusion needle unit of the drug infusion device can be placed in the intermediate position when it leaves the factory. The user does not need to half-press the needle holder to the intermediate position and then perform follow-up operation, which reduces the operation steps requested from the user. As the priming process can be completed during the filling process, avoiding priming waiting, shortening the user's operation period, and significantly improving the user experience.

While the invention has been described in detail regarding the specific embodiments of the present invention, it should be understood that it will be appreciated by those skilled in the art that the above embodiments may be modified without departing from the scope and spirit of the invention. The appended claims define the scope of the invention.

1. A half-press-free drug infusion device, comprising:
a reservoir, configured to accommodate a drug to be infused, provided with a piston and a screw;
a driving wheel, connected with the screw, driving the screw to push the piston forward by rotation;
an infusion needle unit, including an infusion needle and an infusion needle holder, wherein the infusion needle holder is used to keep the infusion needle in place, when the infusion needle unit is at an intermediate position, the infusion needle is communicated with the reservoir;
an adhesive patch, for attaching the half-press-free drug infusion device to a skin surface; and
a security base, arranged on a bottom of the adhesive patch, provided with an infusion needle accommodating part, which is waterproof and breathable.

2. The half-press-free drug infusion device of claim 1, wherein
the security base includes an elastic seal, a waterproof and breathable membrane and a security base body.

3. The half-press-free drug infusion device of claim 2, wherein
the elastic seal and the security base body are integrally formed by injection molding.

4. The half-press-free drug infusion device of claim 2 wherein
the elastic sealing member and the security base body are sealed connected through an interference fit.

5. The half-press-free drug infusion device of claim 3, wherein
the waterproof and breathable membrane is fixed and sealed with the security base body by laser welding or a waterproof double-sided tape.

6. The half-press-free drug infusion device of claim 1, wherein
the security base is a hollow elastic sealing member, at least one side of the hollow elastic sealing member is provided with a plurality of dense micro-holes.

7. The half-press-free drug infusion device of claim 1, wherein
a thickness of the security base is gradually decreased from an end close to one end of the adhesive patch to a middle part of the adhesive patch, and forms a plane with an other end of the adhesive patch.

8. The half-press-free drug infusion device of claim 1, wherein
a thickness of the security base is uniform.

9. The half-press-free drug infusion device of claim 1, wherein
the adhesive patch includes at least one layer of tape and a protective film, the protective film is fixed around an outer contour of the at least one layer of the tape.

10. The half-press-free drug infusion device of claim 1, wherein
the adhesive patch includes at least two layers of tape.

11. The half-press-free drug infusion device of claim 10, wherein
the adhesive patch further includes at least one layer of protective film.

12. The half-press-free drug infusion device of claim 9, wherein
a Rockwell hardness of the protective film is greater than a Rockwell hardness of the tape.

13. The half-press-free drug infusion device of claim 1, wherein
the adhesive patch includes a tape and a release paper, a surface of the tape is provided with dense ventilation holes.

14. The half-press-free drug infusion device of claim 1, wherein
the security base is provided with a through hole.

15. The half-press-free drug infusion device of claim 14, wherein
the through hole is tapered.

16. The half-press-free drug infusion device of claim 1,
further comprising an infusion mechanism module and a control mechanism module, the drug reservoir, the drive wheel, and the infusion needle unit are arranged on the infusion mechanism module.

17. The half-press-free drug infusion device of claim 16, wherein
the infusion mechanism module and the control mechanism module are detachable to each other, and the control mechanism module is reusable.

18. The half-press-free drug infusion device of claim 17, wherein
the infusion mechanism module and the control mechanism module are electrically connected by an electrical contact.

19. The half-press-free drug infusion device of claim 16, wherein
the infusion mechanism module and the control mechanism module are disposed in one housing, discarded together after a single-use.

20. The half-press-free drug infusion device of claim 4, wherein
the waterproof and breathable membrane is fixed and sealed with the security base body by laser welding or a waterproof double-sided tape.
